(19) United States
(12) Patent Application Publication
(10) Pub. No.: US 2024/0226035 A1
(43) Pub. Date: Jul. 11, 2024

(54) Precision Medicine Approach to Treating Parkinson's Disease with a Monoamine Oxidase Inhibitor, an Antioxidant, or Both

(71) Applicant: UNIVERSITY OF NORTH TEXAS HEALTH SCIENCE CENTER AT FORT WORTH, Fort Worth, TX (US)

(72) Inventor: O'Bryant Sid E., Aledo, TX (US)

(21) Appl. No.: 18/559,087

(22) PCT Filed: May 4, 2022
(86) PCT No.: PCT/US2022/027624

§ 371 Date: Nov. 6, 2023

Related U.S. Application Data
(60) Provisional application No. 63/185,544, filed on May 7, 2021.

(51) Int. Cl. A61K 31/137; A61K 31/015; A61K 31/07; A61K 31/095; A61K 31/122; A61K 31/135; A61K 31/138; A61K 31/15; A61K 31/194; A61K 31/352; A61K 31/375; A61K 31/4045; A61K 31/42; A61K 31/421; A61K 31/4985; A61K 31/50; A61K 31/5375; A61K 33/00; A61K 33/04; A61K 38/06; G16B 25/10; G16B 40/20

(52) U.S. Cl. CPC A61K 31/137; A61K 31/015; A61K 31/07; A61K 31/095; A61K 31/122; A61K 31/135; A61K 31/138; A61K 31/15; A61K 31/194; A61K 31/352; A61K 31/375; A61K 31/4045; A61K 31/42; A61K 31/421; A61K 31/4985; A61K 31/50; A61K 31/5375; A61K 33/00; A61K 33/04; A61K 38/063; G16B 25/10; G16B 40/20

(57) ABSTRACT

The present invention includes a method for selecting a subject for a targeted treatment for Parkinson's Disease, the method comprising: obtaining or having obtained a sample from the subject; measuring in the blood or serum sample the expression level of one or more first biomarkers; comparing the level of expression of the one or more first biomarkers from the subject's sample with the expression level of a corresponding one or more first biomarkers from a statistical sample representative of biomarkers in a normal subject; and if the subject is found to have a statistically different expression level of the one or more biomarkers when compared to the expression level of the corresponding one or more biomarkers from the statistical sample, treating the subject with a monoamine oxidase inhibitor, an antioxidant, or both.

### TECHNICAL FIELD OF THE INVENTION

The present invention relates in general to the field of treatments for Parkinson's Disease, and more particularly, to a precision medicine approach to treating Parkinson's Disease with a monoamine oxidase inhibitor, an antioxidant, or both.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with treatments for Parkinson's Disease.

Parkinson's disease (PD) is the second most common neurodegenerative disease affecting over 1% of people age 65 and over in the United States (U.S.)¹. The cost of PD was reported to be $23 billion annually in the U.S. in 20052. Considering the estimated 15% growth in the elderly U.S. population during the last decade, these costs can be expected to increase dramatically as the population ages. Despite advancements in the understanding of PD, current therapeutics remain limited with no disease modifying therapies currently available. In order to advance novel therapeutics in PD, the identification of biomarkers for PD is of the utmost importance³.

While much of today's pharmacotherapy is “trial and error”⁵, precision medicine is a biomarker-guided medicine⁶ that is designed to improve early and accurate diagnostics and therapeutics. The FDA defines precision medicine (also known as “personalized medicine”) as “an innovative approach to tailoring disease prevention and treatment to take into account differences in people's genes, environments, and lifestyles. The goal of precision medicine is to target the right treatment to the right patients at the right time”⁷. In fact, biomarker guided therapies in oncology have resulted in drastically improved patient outcomes⁸. A precision medicine approach has been proposed for numerous diseases⁹; however, few studies have provided direct empirical support.

Despite these advances, a need remains for a robust, comprehensive, reliable methods for identifying PD patient subpopulations that may benefit from certain treatments, while the same patient may not benefit from a treatment, or in which such a treatment would be contraindicated.

### SUMMARY OF THE INVENTION

In one embodiment, the present invention includes a method for selecting a subject for a targeted treatment for Parkinson's Disease (PD), the method comprising: obtaining or having obtained a blood, plasma or serum sample from the subject; measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP); comparing the level of expression of the one or more first biomarkers from the subject's sample with the expression level of a corresponding one or more first biomarkers from a statistical sample representative of biomarkers in a normal subject; and if the subject is found to have a statistically different expression level of the one or more biomarkers when compared to the expression level of the corresponding one or more biomarkers from the statistical sample, treating the subject with a monoamine oxidase inhibitor, an antioxidant, or both. In one aspect, the subject is categorized as a responder or other, wherein those subjects categorized as other include a non-responder or an adverse responder, wherein only a responder is treated with the monoamine oxidase inhibitor, the antioxidant, or both. In another aspect, the monoamine oxidase inhibitor is selected from at least one of: hydrazine, isocarboxazid, hydracarbazine, phenelzine, tranylcypromine, bifemelane, moclobemide, pirlindole, toloxatone, rasagiline, selegiline, safinamide, linezolid, or methylene blue, and salts and combinations thereof. In another aspect, the method further comprises, if the subject has the statistically different expression level of the one or more first biomarkers the subject will need treatment with L-DOPA in 15 months or less. In another aspect, the method further comprises, if the subject has the statistically different expression level of the one or more first biomarkers treating the subject with the monoamine oxidase inhibitor, the antioxidant, or both to delay a treatment with L-DOPA. In another aspect, the method further comprises measuring one or more second biomarkers in the blood, plasma or serum sample, wherein a second statistically different expression level of one or more second biomarkers selected from the group consisting of: Aβ40, Aβ42, total-tau, Neurofilament Light (Nf-L) and α-synuclein is indicative that the subject will respond to a treatment with the monoamine oxidase inhibitor, the antioxidant, or both. In another aspect, the antioxidant is selected from at least one of: ascorbic acid, phenolic acids, sorbic acid, sodium bisulfite, sodium metabisulfite, acetyl cysteine, sodium thiosulfate, ethylene diamine tetraacetic acid, sodium nitrite, ascorbyl stearate, ascorbyl palmitate, sodium ascorbate, monosterol citrate, alpha-thioglycerol, erythorbic acid, cysteine hydrochloride, citric acid, vitamin A, beta-carotene, tocopherol or vitamin E, tocopherol acetate, dibutylhydroxytoluene, soybean lecithin, sodium thioglycolate, butylhydroxyanisole, propyl gallate, tertiary butylhydroquinone, butylated hydroxyanisole, uric acid, cysteine, glutathione, lipoic acid, anthocyanidins, co-enzyme Q10, selenium, melatonin, thiourea, and salts and combinations thereof. In another aspect, the method further comprises the step of delaying a treatment for Parkinson's disease with levodopa. In another aspect, the method further comprises avoiding, not commencing, or discontinuing a treatment for Parkinson's disease, wherein the treatment is providing levodopa. In another aspect, the one or more first biomarkers are selected in order. In another aspect, the expression level of 2, 3, 4, 5, 6, 7, 8, 9 10, 11, 12, 13, 14, 15, 16, 17, or 18 of the first biomarker are measured. In another aspect, the subject responds to an anti-oxidant treatment to delay a need for levodopa for 15 months if two or more first biomarkers are preferentially detected in the following order: eotaxin 3, Factor VII, alpha-2-microglobulin (A2M), IL-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP3). In another aspect, the subject responds to an monoamine oxidase inhibitor treatment to delay a need for levodopa for 15 months if two or more first biomarkers are preferentially detected in the following order: thrombopoietin (TPO), soluble intracellular adhesion molecule (sICAM-1), C-reactive protein (CRP), IL-5, fatty acid binding protein (FABP3), IL-6, soluble vascular cell adhesion molecule 1 (sVCAM-1), alpha-2-microglobulin (A2M), serum amyloid A1 cluster (SAA), IL-7, IL-10, tumor necrosis factor alpha (TNFα), IL-18, tenacin C, eotaxin 3, Chemokine (C-C Motif) Ligand 17 (TARC), inflammatory cytokine I-309 (I-309), beta-2-microglobulin (B2M), and Factor VII. In another aspect, the subject responds to a monoamine oxidase inhibitor and an antioxidant treatment to delay a need for levodopa for 15 months if two or more first biomarkers are preferentially detected in the following order: thrombopoietin (TPO), IL-10, tenacin C, IL-6, serum amyloid A1 cluster (SAA), C-reactive protein (CRP), IL-5, beta-2-microglobulin (B2M), soluble intracellular adhesion molecule (sICAM-1), inflammatory cytokine I-309 (I-309), IL-18, fatty acid binding protein (FABP3), Factor VII, eotaxin 3, tumor necrosis factor alpha (TNFα), alpha-2-microglobulin (A2M), soluble vascular cell adhesion molecule 1 (sVCAM-1), IL-7, and Chemokine (C-C Motif) Ligand 17 (TARC).

In another embodiment, the present invention includes a method for selecting a subject for a targeted treatment for Parkinson's disease, the method comprising: measuring, in a blood, plasma or serum sample obtained from the subject, an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP); and if the subject has a modified expression of the one or more first biomarkers determining if the subject is categorized a responder, or other, wherein other is a non-responder or adverse responder, wherein if the subject is categorized as responder then treating the subject with a monoamine oxidase inhibitor, an antioxidant, or both, or if the patient is categorized as other, then treating the subject with a PD therapy. In one aspect, the monoamine oxidase inhibitor is selected from at least one of: hydrazine, isocarboxazid, hydracarbazine, phenelzine, tranylcypromine, bifemelane, moclobemide, pirlindole, toloxatone, rasagiline, selegiline, safinamide, linezolid, or methylene blue, and salts and combinations thereof. In another aspect, the antioxidant is selected from at least one of: ascorbic acid, phenolic acids, sorbic acid, sodium bisulfite, sodium metabisulfite, acetyl cysteine, sodium thiosulfate, ethylene diamine tetraacetic acid, sodium nitrite, ascorbyl stearate, ascorbyl palmitate, sodium ascorbate, monosterol citrate, alpha-thioglycerol, erythorbic acid, cysteine hydrochloride, citric acid, vitamin A, beta-carotene, tocopherol or vitamin E, tocopherol acetate, dibutylhydroxytoluene, soybean lecithin, sodium thioglycolate, butylhydroxyanisole, propyl gallate, tertiary butylhydroquinone, butylated hydroxyanisole, uric acid, cysteine, glutathione, lipoic acid, anthocyanidins, co-enzyme Q10, selenium, melatonin, thiourea, and salts and combinations thereof. In another aspect, the method further comprises, if the subject has a modified expression of the one or more first biomarkers and the subject is a non-responder or adverse responder the subject will need treatment with L-DOPA in 15 months or less. In another aspect, the method further comprises, if the subject has a modified expression of the one or more first biomarkers and the subject is a responder then treatment with the monoamine oxidase inhibitor, the antioxidant, or both is provided to delay a treatment with L-DOPA. In another aspect, the method further comprises measuring one or more second biomarkers in the blood, plasma or serum sample, wherein a modified expression level of the one or more second biomarkers selected from the group consisting of: Aβ40, Aβ42, total-tau, Neurofilament Light (Nf-L) and α-synuclein is indicative that the subject will respond to a treatment with the monoamine oxidase inhibitor, the antioxidant, or both. In another aspect, the method further comprises measuring in the blood or serum sample the expression level of one or more second biomarkers selected from the group consisting of: Aβ40, Aβ42, total-tau, Neurofilament Light (Nf-L) and α-synuclein. In another aspect, the method further comprises avoiding, not commencing, or discontinuing the treatment for Parkinson's disease with the monoamine oxidase inhibitor, the antioxidant, or both, and then providing levodopa. In another aspect, the method further comprises avoiding, not commencing, or discontinuing a treatment for Parkinson's disease, wherein the treatment is providing levodopa. In another aspect, the one or more first biomarkers are selected in order. In another aspect, the expression level of 2, 3, 4, 5, 6, 7, 8, 9 10, 11, 12, 13, 14, 15, 16, 17, or 18 of the first biomarker are measured. In another aspect, the subject responds to an anti-oxidant treatment to delay a need for levodopa for 15 months if two or more first biomarkers are preferentially detected in the following order: eotaxin 3, Factor VII, alpha-2-microglobulin (A2M), IL-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP3). In another aspect, the subject responds to an monoamine oxidase inhibitor treatment to delay a need for levodopa for 15 months if two or more first biomarkers are preferentially detected in the following order: thrombopoietin (TPO), soluble intracellular adhesion molecule (sICAM-1), C-reactive protein (CRP), IL-5, fatty acid binding protein (FABP3), IL-6, soluble vascular cell adhesion molecule 1 (sVCAM-1), alpha-2-microglobulin (A2M), serum amyloid A1 cluster (SAA), IL-7, IL-10, tumor necrosis factor alpha (TNFα), IL-18, tenacin C, eotaxin 3, Chemokine (C-C Motif) Ligand 17 (TARC), inflammatory cytokine I-309 (I-309), beta-2-microglobulin (B2M), and Factor VII. In another aspect, the subject responds to a monoamine oxidase inhibitor and an antioxidant treatment to delay a need for levodopa for 15 months if two or more first biomarkers are preferentially detected in the following order: thrombopoietin (TPO), IL-10, tenacin C, IL-6, serum amyloid A1 cluster (SAA), C-reactive protein (CRP), IL-5, beta-2-microglobulin (B2M), soluble intracellular adhesion molecule (sICAM-1), inflammatory cytokine I-309 (I-309), IL-18, fatty acid binding protein (FABP3), Factor VII, eotaxin 3, tumor necrosis factor alpha (TNFα), alpha-2-microglobulin (A2M), soluble vascular cell adhesion molecule 1 (sVCAM-1), IL-7, and Chemokine (C-C Motif) Ligand 17 (TARC).

In another embodiment, the present invention includes a method of identifying a Parkinson's Disease patient that will respond to a treatment that delays the need for L-DOPA comprising: obtaining a blood, plasma or serum sample from the patient; measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP); comparing the level of expression from the blood, plasma or serum sample with a statistical sample representative of a normal subject; and if the patient has a statistically different expression level of the one or more biomarkers when compared to the expression level of a corresponding one or more biomarkers from the statistical sample, treating the patient with the monoamine oxidase inhibitor, the antioxidant, or both to delay the treatment with L-DOPA.

In another embodiment, the present invention includes a method of predicting and delaying or treating a loss of motor function, a loss of cognition, or both in a PD patient response to therapy comprising: obtaining or having obtained a blood, plasma or serum sample from the patient; measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP); comparing the level of expression from the sample with a statistical sample representative of a normal subject; determining if the patient has a statistically different expression level of the one or more biomarkers when compared to the expression level of a corresponding one or more biomarkers from the statistical sample; and treating the patient having the statistically different expression level of the one or more biomarkers with a monoamine oxidase inhibitor, an antioxidant, or both, to delay or treat the loss of motor function, the loss of cognition, or both.

In another embodiment, the present invention includes a method of identifying a subject with Parkinson's disease (PD) who will not respond to a monoamine oxidase inhibitor therapy, an antioxidant therapy, or a combination thereof, the method comprising: obtaining or having obtained a blood, plasma or serum sample from the subject; measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP); comparing the level of expression from the sample with a statistical sample representative of a normal subject; determining if the subject has a statistically different expression level of the one or more biomarkers when compared to the expression level of a corresponding one or more biomarkers from the statistical sample, wherein the statistically different expression level is indicative that the subject is categorized as a responder to a treatment with the monoamine oxidase inhibitor therapy, the antioxidant therapy, or a combination thereof, or the patient is categorized as a non-responder or an adverse responder; and if the subject is categorized as non-responder or adverse responder, treating the subject with a traditional PD therapy selected from levodopa, carbidopa-levodopa, dopamine agonists, catechol O-methyltransferase inhibitors, anticholinergics, or amantadine.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C are Confusion matrix and variable importance plot for treatment arm receiving TOC for the full MSD panel for distinguishing primary and secondary endpoints.

FIGS. 2A to 2C are Confusion matrix and variable importance plot for treatment arm receiving DEP for the full MSD panel for distinguishing primary and secondary endpoints.

FIGS. 3A to 3C are Confusion matrix and variable importance plot for the combined treatment arm receiving both TOC and DEP for the full MSD panel for distinguishing primary and secondary endpoints.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “Parkinson's Disease patient”, and “individual diagnosed with Parkinson's disease” all refer to an individual who has been diagnosed with PD or has been given a diagnosis of Parkinson's disease.

As used herein, the terms “cognition”, “cognitive ability”, “memory”, “language” and the like are used interchangeably to refer to an individual's ability to perform cognitive abilities and the dysfunction of those abilities that may be as a result of a diagnosis of PD, MCI, AD, DLB, FTD, DLB, Multiple Sclerosis (MS), or other neurological disease as well as other medical and psychiatric conditions including, but not limited to, diabetes, hypertension, dyslipidemia, metabolic syndrome, depression, traumatic brain injury, schizophrenia, bipolar disease, as well as the cognitive slowing/decline associated with the aging process itself.

As used herein, the term “predicting” refers to making a finding that an individual has a significantly enhanced probability of responding or not responding to a therapy.

As used herein, “biological fluid sample” refers to a wide variety of fluid sample types obtained from an individual and can be used in a diagnostic or monitoring assay. Biological fluid sample include, e.g., blood, plasma, serum, cerebral spinal fluid (CSF), urine and other liquid samples of biological origin. Commonly, the samples are treatment with stabilizing reagents, solubilization, or enrichment for certain components, such as proteins or polynucleotides, so long as they do not interfere with the analysis of the markers in the sample.

As used herein, a “blood sample” refers to a biological sample derived from blood, preferably peripheral (or circulating) blood. A blood sample may be, e.g., whole blood, serum or plasma. In certain embodiments, serum is preferred as the source for the biomarkers as the samples are readily available and often obtained for other sampling, is stable, and requires less processing, thus making it ideal for locations with little to refrigeration or electricity, is easily transportable, and is commonly handled by medical support staff.

As used herein, the terms “normal,” “control,” and “healthy,” refer generally to a subject or individual who does not have, is not, and/or has not been diagnosed with, and/or is asymptomatic for a particular disease or disorder, specifically, a neurological disorder, or one or more symptoms of motor function loss, cognitive loss, or Parkinson's Disease. As such, a normal subject is a subject that does not have a neurodegenerative disease. The terms can also refer to a sample obtained from such subject or individual. The disease or disorder under analysis or comparison is determinative of whether the subject is a “control” in that situation and for that disease or disorder. For example, where the level of a particular biomarker is obtained from an individual known to have motor function loss, cognitive loss, or Parkinson's Disease, that subject can be the “PD subject.” The level of the marker thus obtained from the PD subject can be compared to a level of that same biomarker from a subject who is not diagnosed with motor function loss, cognitive loss, or Parkinson's Disease and who is known not to have prevalent motor function loss, cognitive loss, or Parkinson's Disease, i.e., a “normal subject.” Thus, “normal subject” in this example refers to a subject that does not have any neurological disorders, motor function loss, cognitive loss, or Parkinson's Disease. A “normal” individual or a sample from a “normal” individual can also refer to quantitative data, qualitative data, or both from an individual who has or would be assessed by a physician as not having a disease, e.g., a neurological disease. Often, a “normal” individual is also age-matched within a range of 1, 2, 3, 4, 5, 6, 7, 8, 9 or 10 years with the sample of the individual to be assessed.

As used herein, the term “treatment” refers to the alleviation, amelioration, and/or stabilization of symptoms, as well as delay in progression of symptoms of a particular disorder. For example, “treatment” of PD includes any one or more of: (1) elimination of one or more symptoms of PD, (2) reduction of one or more symptoms of PD, (4) stabilization of the symptoms of PD (e.g., failure to progress to more advanced stages of PD), and (5) delay in onset of one or more symptoms of PD delay in progression (i.e., worsening) of one or more symptoms of PD; and (6) delay in progression (i.e., worsening) of one or more symptoms of PD.

As used herein, a “statistical sample representative of the subject” or a “statistical sample representative of the patient” refers to a statistical sample comprising one or more of the following groups of individuals: (1) individuals with a family history of motor function loss, cognitive loss, or Parkinson's Disease; (2) individuals with no family history of motor function loss, cognitive loss, or Parkinson's Disease; (3) individuals who have motor function loss, cognitive loss, or Parkinson's Disease; and (4) individuals who do not have Parkinson's Disease.

In some embodiments, the statistical sample comprises elderly individuals. In some embodiments, the individuals are 30 years of age or older. In some embodiments, the data obtained from the patient or subject (e.g., demographic factors, neurocognitive evaluation results, and biomarker expression level) are compared to the corresponding data from individuals in the statistical sample. This comparison is discussed elsewhere herein and can be applied to any method of the present disclosure.

As used herein, a “monoamine oxidase inhibitor” refers to molecules or agents that inhibit the activity of monoamine oxidase. Non-limiting examples of monoamine oxidase inhibitor(s) include: hydrazine, isocarboxazid, hydracarbazine, phenelzine, tranylcypromine, bifemelane, moclobemide, pirlindole, toloxatone, rasagiline, selegiline, safinamide, linezolid, or methylene blue, and salts and combinations thereof. In some embodiments, the monoamine oxidase inhibitor is present in an amount of 0.1, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

As used herein, an “antioxidant” refers to molecules or agents that inhibits the oxidation of other molecules. Non-limiting examples of antioxidant(s) include: ascorbic acid, phenolic acids, sorbic acid, sodium bisulfite, sodium metabisulfite, acetyl cysteine, sodium thiosulfate, ethylene diamine tetraacetic acid, sodium nitrite, ascorbyl stearate, ascorbyl palmitate, sodium ascorbate, monosterol citrate, alpha-thioglycerol, erythorbic acid, cysteine hydrochloride, citric acid, vitamin A, beta-carotene, tocopherol or vitamin E, tocopherol acetate, dibutylhydroxytoluene, soybean lecithin, sodium thioglycolate, butylhydroxyanisole, propyl gallate, tertiary butylhydroquinone, butylated hydroxyanisole, uric acid, cysteine, glutathione, lipoic acid, anthocyanidins, co-enzyme Q10, selenium, melatonin, thiourea, and salts and combinations thereof. In some embodiments, the antioxidant is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

As used herein, an “effective amount” refers to a monoamine oxidase inhibitor, an antioxidant, or both, in an amount necessary or sufficient to treat a subject, or to provide improvement, benefit, or enhancement to health or performance. An improvement, benefit, or enhancement to health or performance can include, e.g., preventing or reducing a loss of motor function, a loss of cognition, or reduction or amelioration of symptoms related to PD. It can also include increase the length of time in which a subject does not need additional treatment for symptoms of PD, such as treatment with L-DOPA. The effective amount can vary depending on such factors as the size and weight of the subject, the type of result or outcome, or the particular quercetin compound. For example, the choice of the monoamine oxidase inhibitor, an antioxidant, or both can affect what constitutes an “effective amount”. One of ordinary skill in the art would be able to study the aforementioned factors and make the determination regarding the effective amount of the quercetin compound without undue experimentation.

The present inventor recognized that Parkinson's Disease (PD) is a heterogeneous condition and that a paradigm shift is required to make substantial advancements in therapeutic outcomes. Specifically, the present inventor recognized that specific subsets of PD patients can be treated with targeted therapeutics.

The FDA defines a “predictive biomarker” as “a biomarker used to identify individuals who are more likely than similar patients without the biomarker to experience a favorable or unfavorable effect from a specific intervention or exposure.”¹⁰ Further, the lack of significant advancement in clinical trials targeting PD is due to the “one-size-fits-all” approach.

The role of inflammation in neurodegeneration has received a great deal of attention¹¹⁻¹⁶ and has been linked to PD¹⁷⁻¹⁹ as well as AD^{20, 21}, frontotemporal dementia (FTD)²² and dementia with Lewy bodies (DLB)²³. In fact, it has been suggested that profiling biological pathways associated with multiple neurodegenerative diseases may highlight novel biological pathways for therapeutic interventions for neurodegenerative diseases^{24, 25} and that inflammation may be such a pathway^{18, 26}. Numerous studies have implicated inflammation in PD²⁷⁻²⁹. Neuroinflammation has been found to be significantly upregulated in PD in animal as well as human models³⁰. Additionally, it has been demonstrated that the induction of PD-like symptoms in humanized CD34+ mice using MPTP can cause an associated neuroinflammatory response that was attenuated using FK506, an immunosuppressant drug. Based on these and other studies³¹, it has been suggested that anti-inflammatory medications may have utility in PD³²⁻³⁶. In fact, one recent study demonstrated that probiotic intake impacted inflammatory gene expression in PD patients after 12-weeks³⁷ with another showing that probiotic supplementation reduced inflammation and decreased MDS-UPDRS scores³⁷. Administration of omega-3 fatty acids with vitamin E has been shown to reduce inflammation and improve UPDRS scores in PD patients³⁸. The DATATOP trial found that deprenyl 10 mg/day delayed time until disability that warranted initiation of levodopa therapy but there was no benefit of deprenyl in postponing levodopa-related adverse events or extending life³⁹⁻⁴¹ though problems have been noted with study design and endpoints^{42, 43}.

The present inventor recognized that leveraging previously conducted clinical trial biorepositories, it is possible to demonstrate the precision medicine approach. Here, a predictive group of biomarkers was generated to identify the specific subset of PD patients that will respond to a given therapy. In one example, the present invention is a proteomic-based predictive biomarker that could predict treatment response in the previously conducted DATATOP trial. Using the methods of the present invention it is possible to identify specific subgroups of clinical trial participants that responded to a particular therapy, which can be used to identify the subset of PD participants that benefited most from the DATATOP trial, and to select treatments that will apply to the different subgroups of PD patients, thereby increasing the effectiveness and precision of treatments.

Participants and Methods of Deprenyl and Tocopherol Antioxidative Therapy of Parkinsonism (DATATOP).

The sample included in this study were derived from available biorepository samples from participants enrolled in a previously conducted clinical trial for PD (DATATOP). The DATATOP⁴⁰ trial enrolled a total of 800 participants over a 2-year time frame between September 1987 and November 1988 across multiple sites. Of those participants enrolled in the total DATATOP clinical trial, for the current study, the following trial participants were included for further analysis: n=164 from the a-tocopherol (an antioxidant) and deprenyl (a monoamine oxidase inhibitor) placebo treatment arm, n=185 from the deprenyl and a-tocopherol placebo treatment arm, and n=171 from the combined active deprenyl and a-tocopherol treatment arm. Inclusion criteria for the trial included a diagnosis of PD (Hoehn and Yahr stage as I or II) within the past 5 years⁴⁰. Exclusion criteria included the presence of a severe tremor, dementia diagnosis, depression, or use of a symptomatic PD medication. Participants who met inclusion criteria were randomized into one of the following treatment arms: a-tocopherol (2000 IU/d) and deprenyl placebo (TOC), deprenyl (10 mg/d) and a-tocopherol placebo (DEP), active deprenyl and atocopherol (TOC and DEP), or double placebo⁴⁰. After 14 months, the trial was discontinued due to the positive effects shown among those receiving deprenyl on reducing PD symptoms and the study became an open-label administration for the remainder of the two-year trial⁴⁴.

The DATATOP trial primary endpoint was PD symptom progression suggestive of disability requiring dopaminergic therapy. Secondary endpoints included change in PD symptoms as measured by the Unified Parkinson's Disease Rating Scale (UPDRS) motor and total scores⁴⁰. The UPDRS total score is comprised of the sum of the motor, cognitive, and ADL subscales. Change in UPDRS score was calculated based on prior work⁴⁵. For purposes of this study, the determination of responder status for secondary trial endpoints were made based on empirically defined cut-offs for clinically meaningful change scores in UPDRS motor and UPDRS total scores⁴⁶. For UPDRS motor score, the clinically meaningful cut-off score was less than or equal to −2.5 for responders, while others (non-responders and adverse responders) were defined as those greater than or equal to −2.5⁴⁶. For UPDRS total score, the clinically meaningful cut-off score was less than or equal to −4.3 for responders, while others (non-responders and adverse responders) were defined as those greater than or equal to −4.3⁴⁶. For the primary trial endpoint, those participants who did not require Levodopa at 15 months were classified as responders while those who did require Levodopa at the end of the trail were classified as other (non-responder or adverse responder).

### Proteomic Assays.

All blood biomarker assays were conducted at the University of North Texas Health Science Center in the Institute for Translational Research (ITR) Biomarker Core. Preparation of samples for proteomic assay was conducted using the Hamilton Robotics StarPlus system while any re-aliquoting was conducted via the Hamilton easyBlood robotic system. Plasma samples were assayed via multi-plex biomarker assay platform using electrochemiluminescence (ECL). Plasma samples were assayed with commercially available assays for the following markers in the full MSD panel: CRP, sICAM1, sVCAM1, SAA, IL-6, IL-10, TNF-α, IL-5, IL-7, Eotaxin-3, TARC, A2M, B2M, FVII, TNC, FABP-3, IL-18, TPO and I-309. The ITR lab has assayed over >20,000 samples on these markers using this system. Inter- and intra-assay variability has been excellent^{47, 48}. Average CVs (>3000 samples) for these assays are all <10% with the majority being <=5%. The ultrasensitive Simoa platform was also used to assay the supplemental proteomic panel markers that included plasma Amyloid beta (A3) 40 and 42, total-tau, neurofilament light chain (Nf-L) and alpha-synuclein. More than >5,000 samples were processed using this platform and CVs remain <=5%^{49, 50}.

### Statistical Analyses.

The predictive biomarker profile was generated using support vector machine (SVM) analyses. SVM is based on the concept of decision planes that define decision boundaries and serves as a classifier method by performing classification tasks through constructing hyperplanes in a multidimensional space that can separate cases of different class labels. SVM predictive biomarker profiles were based on responders versus others (non-responders and adverse responders)(i.e., only 2 groups). Treatment responder was defined as UPDRS (motor and total) score that was stable or improved at 15 months from baseline whereas non-responder was defined as any decline in UPDRS scores during this same time point. Treatment responder was also defined as not requiring Levodopa at 15 months while others (non-responders and adverse responders) were those who required Levodopa. The purpose of this approach was to have a predictive biomarker that could selectively identify only those most likely to respond while all others would be ruled out. Internal five-fold crossvalidation was conducted within the sample with the SVM analyses. The SVM analyses were conducted with the e1071 package (v1.6-8) in R (v3.4.2). In order to build an SVM model to predict treatment response, the radial basis function kernel was used together with five-fold cross-validation, cost=100 and gamma=0.001. The original data was randomly partitioned into 5 equal sized subsamples. A single subsample was retained as a testing set and the remaining 4 subsamples were used as training sets. For each model, we ran the cross-validation randomly five times. SVM does not assume normality and, therefore, raw baseline proteomic data were utilized. The analyses were restricted to response.

### TOC Treatment Arm.

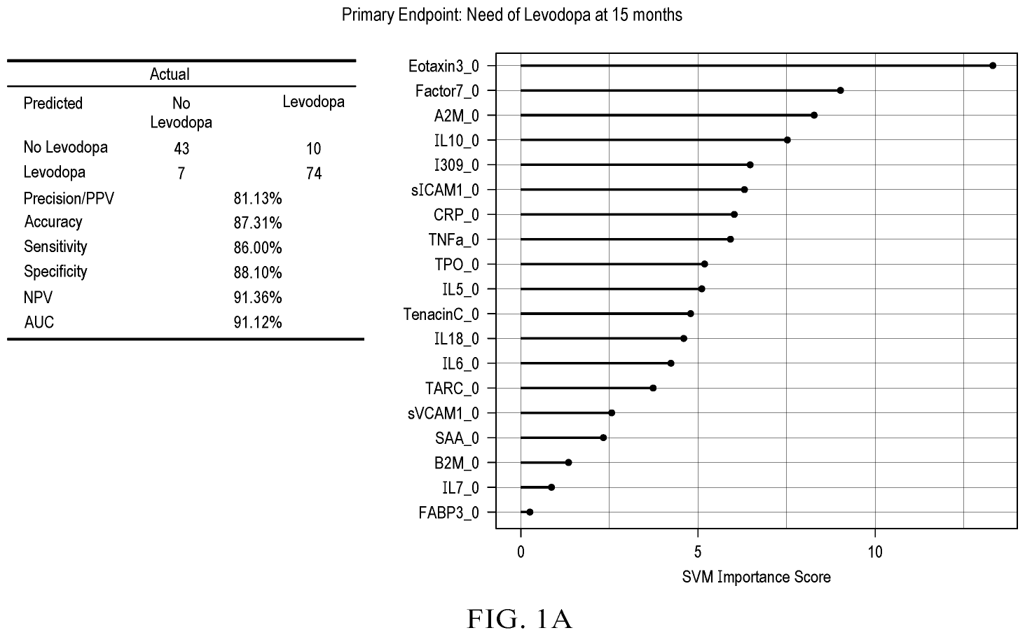

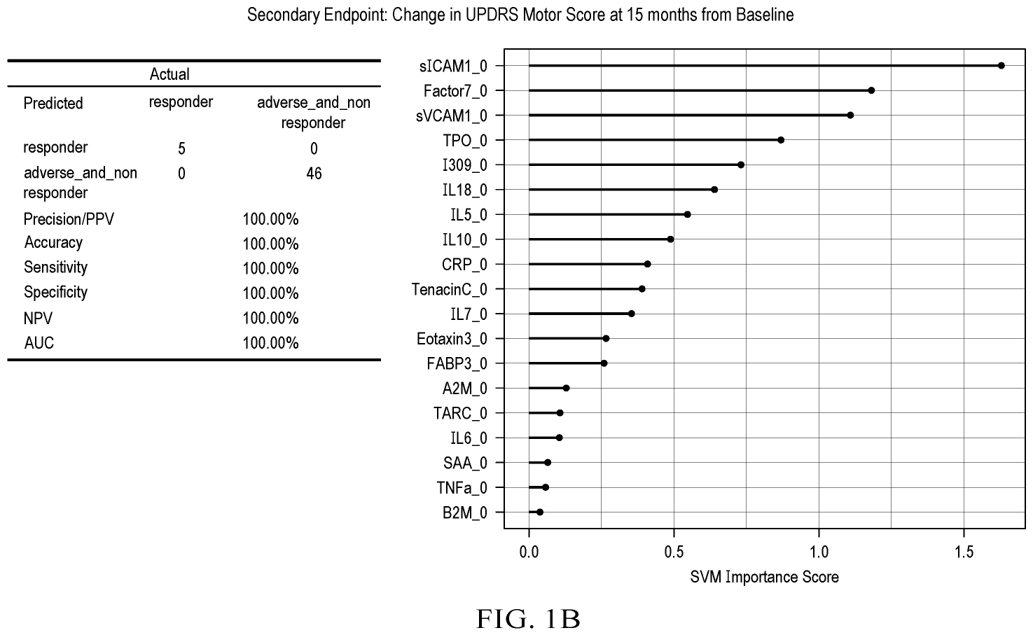

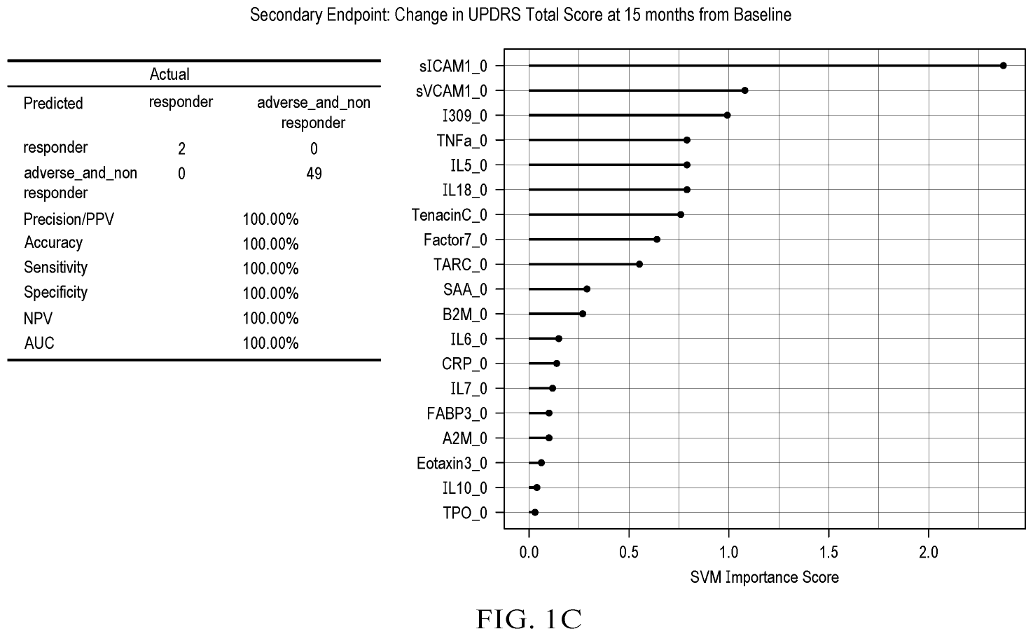

The DATATOP study outlined several endpoints with the primary being the need for Levodopa and the secondary being change in UPDRS motor and total scores. When the full MSD proteomic panel was applied to the primary endpoint for those randomized into the TOC treatment arm, it produced an AUC of 0.91 for distinguishing responder (did not require Levodopa at 15 months) versus non-responder (required Levodopa) with a sensitivity of 0.86 and specificity of 0.88 with an optimized cut-off of 0.557 (FIG. 1A). In the same treatment arm (TOC), when the full MSD proteomic panel was applied to detect the secondary endpoint for change in UPDRS motor scores at 15 months from baseline, AUC, sensitivity, and specificity reached 1.00 for distinguishing those who responded and those who experienced no-response or an adverse response with an optimized cutoff of 0.852 (FIG. 1B). Again, in the same treatment arm (TOC), when applied to change in UPDRS total score from baseline to 15 months, the full MSD proteomic panel reached an AUC, sensitivity, and specificity of 1.00 for distinguishing those who responded and those who experienced no-response or an adverse response with an optimized cut-off of 0.798 (FIG. 1C).

### DEP Treatment Arm.

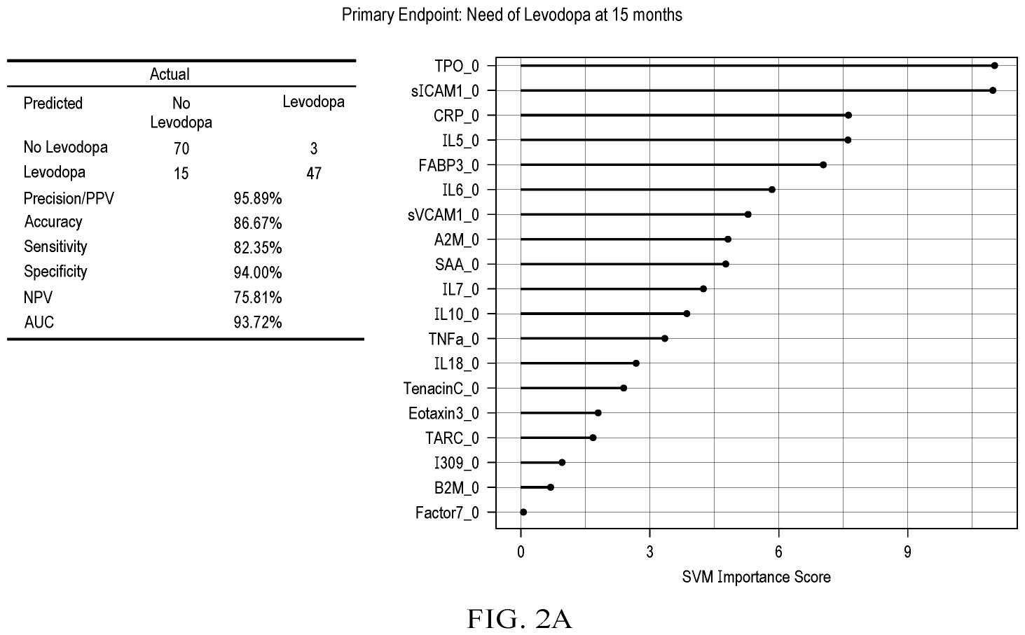

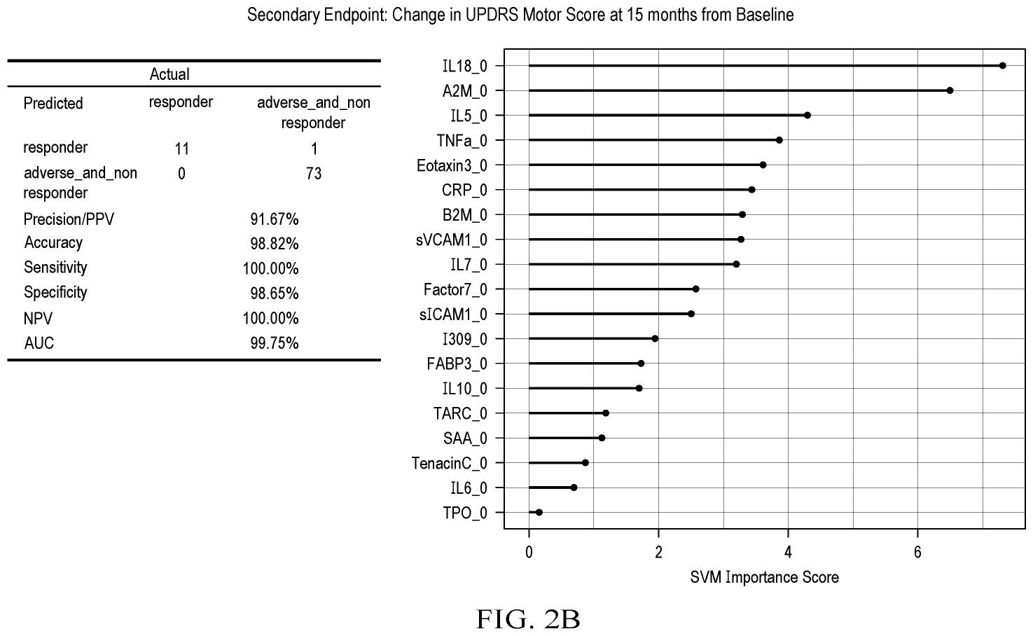

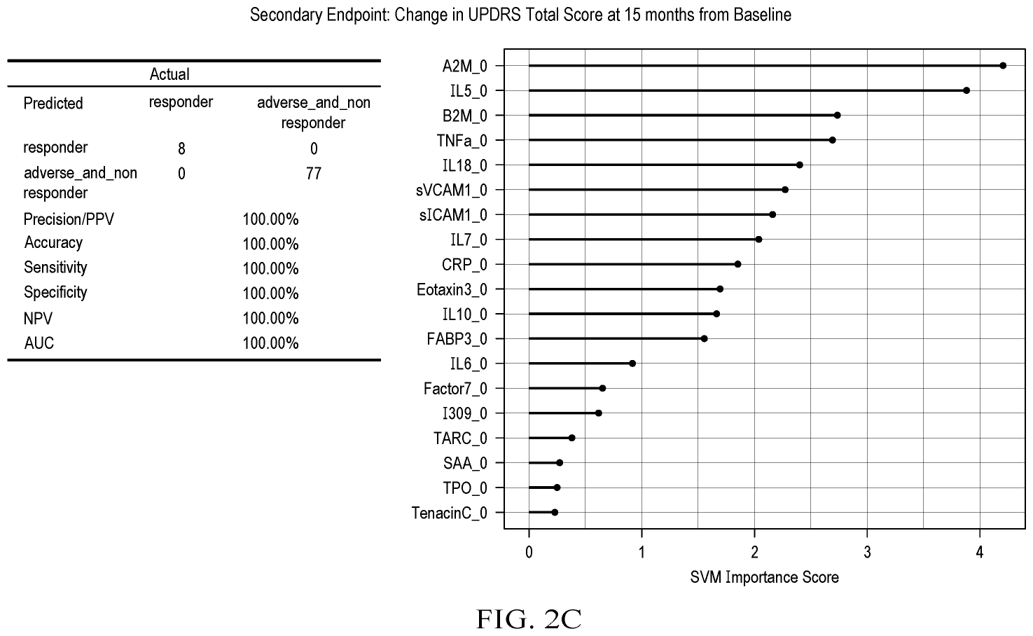

When the full MSD proteomic panel was applied to the primary endpoint for those randomized into the DEP treatment arm, the AUC reached 0.93 while the sensitivity reached 0.82 and specificity reached 0.94 with an optimized cut-off of −0.914 for distinguishing responders versus those who were non-responders or adverse responders (FIG. 2A). When the same proteomic panel was applied to the secondary endpoint of change in UPDRS motor scores at 15 months from baseline in the same treatment arm (DEP), the AUC reached 0.99, while sensitivity and specificity both reached 1.00 in distinguishing responders versus non-responders and adverse responders with an optimized cut-off score of 0.891 (FIG. 2B). When applied to the other secondary endpoint of change in UPDRS total scores at 15 months from baseline again in the same treatment arm (DEP), AUC, sensitivity, and specificity all reached 1.00 with an optimized cut-off of 0.961 (FIG. 2C).

### Combined TOC and DEP Treatment Arm.

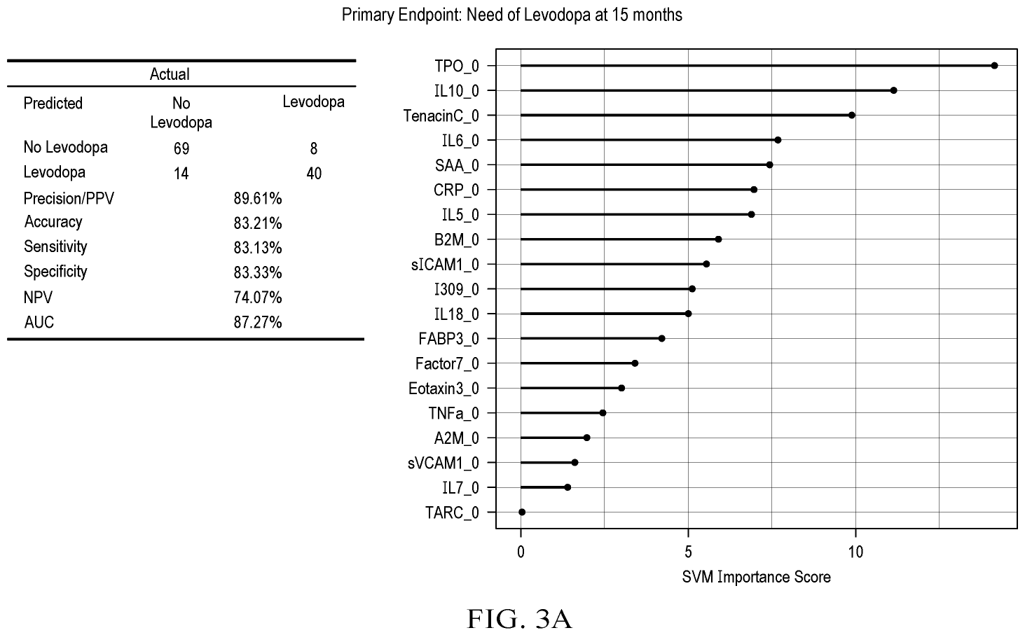

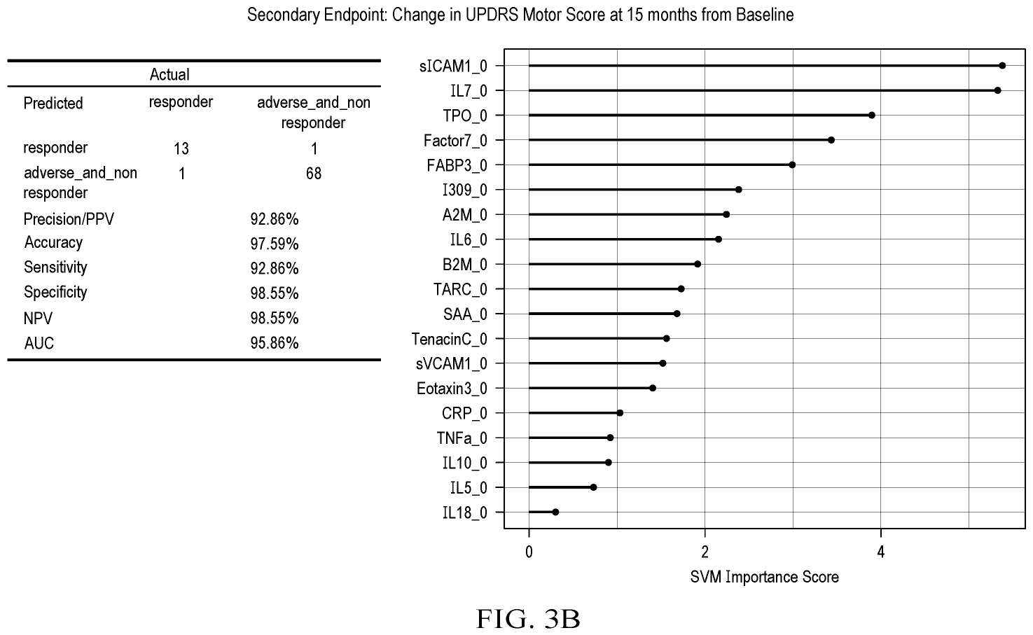

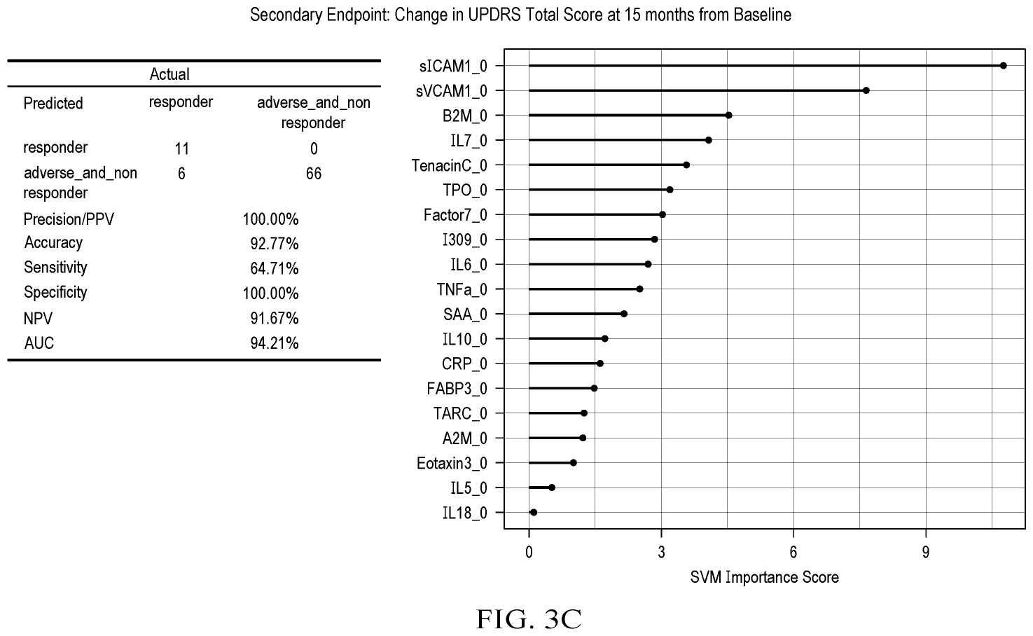

When the full MSD proteomic panel was additionally applied to the primary endpoint for those randomized into the treatment arm of combined TOC and DEP, it produced an AUC of 0.87 with a sensitivity of 0.83 and specificity of 0.83 with an optimized cut-off of −0.881 for distinguishing responders from non-responders and adverse responders (see FIG. 3A). Again, for the combined treatment arm (TOC and DEP), the same proteomic panel remained elevated when applied to the secondary endpoint of change in UPDRS motor scores from baseline to 15 months for distinguishing responders from nonresponders and adverse responders with an AUC of 0.96, sensitivity of 0.93 and specificity of 0.99 with an optimized cut-off of 0.944 (FIG. 3B). When applied to detect another secondary endpoint of change in UPDRS total score from baseline to 15 months for the same combined treatment arm (TOC and DEP), the same proteomic panel reached an AUC of 0.94, sensitivity of 0.64, and specificity of 1.00 with an optimized cut-off of 0.731 for distinguishing responders from non-responders and adverse responders (FIG. 3C).

### Supplemental Proteomic Panel.

Supplemental analyses expanded the MSD proteomic panel to include additional biomarkers specifically related to AD (Aβ40 and 42, total-tau, Nf-L) and PD (α-synuclein) pathology. When this supplemental panel was applied to detect responders from non-responders and adverse responders for those requiring Levodopa at 15 months in the TOC treatment arm, the AUC increased to 0.93 while sensitivity remained unchanged and specificity increased to 0.90 with an optimized cut-off of 0.554. When applied to detect change in UPDRS motor and total scores in this same treatment arm (TOC) at 15 months from baseline, AUC, sensitivity, and specificity remained unchanged with the additional markers. For the DEP treatment arm, the supplemental proteomic panel increased AUC just slightly to 0.95 when applied to distinguish those who required Levodopa at 15 months while sensitivity increased to 0.89 and specificity decreased to 0.92 with an optimized cut-off of −0.863. In this same treatment arm (DEP), the added biomarkers increased AUC and specificity to 1.00 for detecting change in UPDRS motor scores at 15 months from baseline (optimized cut-off for UPDRS motor score of 0.864). AUC, sensitivity, and specificity remained unchanged in this treatment arm for distinguishing responder status for UPDRS total scores. When applied to the combined treatment arm (TOC and DEP), the added biomarkers increased AUC to 0.91, sensitivity to 0.87, and specificity to 0.89 with an optimized cutoff of −0.916 when applied to distinguishing the primary endpoint for need of Levodopa at 15 months. For distinguishing responder status in this same combined treatment arm (TOC and DEP) based on change in UPDRS motor score from baseline to 15 months, the added biomarkers increased AUC, along with sensitivity and specificity to 1.00 with an optimized cut-off of 0.917. Similarly, for this same treatment arm (TOC and DEP), the supplemental proteomic panel increased AUC and sensitivity for distinguishing change in UPDRS total score from baseline to 15 months while specificity remained unchanged at 1.00 with an optimized cut-off of 0.9.

The current results provide strong empirical support for the investigation into a precision medicine approach to treating patients suffering from Parkinson's disease (PD). These results revealed that the predictive biomarker was able to accurately identify responder status for the primary endpoint with AUCs ranging from 87-93%. Higher prediction accuracy was found when applying the same predictive biomarker for distinguishing responder status on secondary trial endpoints (UPDRS motor and total scores) with AUCs ranging from 94-100% with the applied optimized cut-off score. The addition of AD and PD specific biomarkers to the supplemental predictive biomarker increased detection accuracy only slightly (AUC increase of 2%) across treatment arms for the primary endpoint as well as for the secondary endpoint for those in the DEP (UPDRS motor score AUC increase of 1%) and combined TOC and DEP (UPDRS motor score AUC increase of 5%, UPDRS total score AUC increase of 6%) treatment arms. Across treatment arms, the variable importance plots revealed a number of inflammatory proteins among the top markers when applied to trial endpoints.

The inventor's prior work demonstrates that inflammatory markers are highly relevant in detecting and discriminating PD from other neurodegenerative diseases. This work did not produce results helpful for screening or excluding a patent from further testing, but only at detecting and discriminating PD from other neurodegenerative diseases. A study was conducted in the Harvard Biomarker Study Biorepository to assess the ability to discriminate PD from other neurodegenerative diseases by assaying n=150 plasma samples (PD n=50, “other neurodegenerative disease” n=50 [AD n=12, FTD n=25, other n=13], controls n=50)⁴⁹. The proteomic profile approach was highly accurate in discriminating PD from other neurodegenerative diseases with an AUC=0.98.

Additionally, multiple inflammatory markers were among the top 10 in the profile for discriminating PD from other neurodegenerative disease⁴⁹. In a combined analysis of n>800 PD samples and n>2000 controls samples, the inventor's PD Blood Test to detect or differentiate PD from other neurological diseases for primary care practices was heavily weighted for inflammatory markers. This earlier work did not identify methods for predicting how a patient might respond or not respond to a therapy.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Tysnes O B, Storstein A. Epidemiology of Parkinson's disease. J Neural Transm. 2017; 124(8):901-905. doi:10.1007/s00702-017-1686-y.
2. Huse D M, Schulman K, Orsini L, Castelli-Haley J, Kennedy S, Lenhart G. Burden of illness in Parkinson's disease. Mov Disord. Published online 2005. doi:10.1002/mds.20609.
3. Gerlach M, Maetzler W, Broich K, et al. Biomarker candidates of neurodegeneration in Parkinson's disease for the evaluation of disease-modifying therapeutics. J Neural Transm. Published online 2012. doi:10.1007/s00702-011-0682-x.
4. O'Bryant S E, Zhang F, Johnson L A, et al. A Precision Medicine Model for Targeted NSAID Therapy in Alzheimer's Disease. J Alzheimers Dis. 2018; 66(1):97-104. doi: 10.3233/JAD-180619.
5. Jorgensen J T. Companion diagnostics: the key to personalized medicine. Foreword. Expert Rev Mol Diagn. 2015; 15(2):153-156. doi:10.1586/14737159.2015.1002470.
6. Hampel H, O'Bryant S E, Castrillo J I, et al. PRECISION MEDICINE—The Golden Gate for Detection, Treatment and Prevention of Alzheimer's Disease. J Prev Alzheimer's Dis. Published online 2016. doi:10.14283/jpad.2016.112.
7. Precision Medicine|FDA. Accessed Dec. 20, 2020. www.fda.gov/medical-devices/vitro-diagnostics/precision-medicine.
8. Jorgensen J T. Clinical application of companion diagnostics. Trends Mol Med. 2015; 21(7):405-407. doi:10.1016/j.molmed.2015.05.003.
9. Cresci S, Depta J P, Lenzini P A, et al. Cytochrome p450 gene variants, race, and mortality among clopidogrel-treated patients after acute myocardial infarction. Circ Cardiovasc Genet. Published online 2014. doi:10.1161/CIRCGENETICS.113.000303.
10. FDA-NIH Biomarker Working Group. BEST (Biomarkers, EndpointS, and Other Tools) Resource Glossary; 2016. doi:10.1093/neuonc/nox242.
11. Komulainen P, Lakka T A, Kivipelto M, et al. Serum high sensitivity C-reactive protein and cognitive function in elderly women. Age Ageing. Published online 2007. doi:10.1093/ageing/afm051.
12. Schmidt M F, Freeman K B, Windham B G, et al. Associations Between Serum Inflammatory Markers and Hippocampal Volume in a Community Sample. J Am Geriatr Soc. 2016; 64(9):1823-1829. doi:10.1111/jgs.14283.
13. Newman A B, Fitzpatrick A L, Lopez O, et al. Dementia and Alzheimer's disease incidence in relationship to cardiovascular disease in the cardiovascular health study cohort. J Am Geriatr Soc. Published online 2005. doi:10.1111/j.1532-5415.2005.53360.x.
14. Engelhart M J, Geerlings M I, Meijer J, et al. Inflammatory Proteins in Plasma and the Risk of Dementia: The Rotterdam Study. Arch Neurol. 2004; 61(5):668-672. archneur.ama-assn.org/cgi/content/abstract/61/5/668.
15. Wright C B, Sacco R L, Rundek T R, Delman J B, Rabbani L E, Elkind M S V. Interleukin-6 is associated with cognitive function: The Northern Manhattan Study. J Stroke Cerebrovasc Dis. Published online 2006. doi:10.1016/j.jstrokecerebrovasdis.2005.08.009.
16. Yaffe K, Lindquist K, Penninx B W, et al. Inflammatory markers and cognition in well-functioning African-American and white elders. Neurology. 2003; 61(1):76-80.
17. Akil E, Bulut A, Kaplan I, zdemir H H, Arslan D, Alu.lu M U. The increase of carcinoembryonic antigen (CEA), high-sensitivity C-reactive protein, and neutrophil/lymphocyte ratio in Parkinson's disease. Neurol Sci. Published online 2015. doi:10.1007/s10072-014-1976-1.
18. Durrenberger P F, Fernando F S, Kashefi S N, et al. Common mechanisms in neurodegeneration and neuroinflammation: a BrainNet Europe gene expression microarray study. J Neural Transm. Published online 2015. doi:10.1007/s00702-014-1293-0.
19. Cebrin C, Zucca F A, Mauri P, et al. MHC-I expression renders catecholaminergic neurons susceptible to T-cell-mediated degeneration. Nat Commun. Published online 2014. doi:10.1038/ncomms4633.
20. O'Bryant S E, Johnson L, Edwards M, et al. The link between c-reactive protein and Alzheimer's disease among Mexican Americans. J Alzheimer's Dis. Published online 2013. doi:10.3233/JAD-122071.
21. Holmes C, Butchart J. Systemic inflammation and Alzheimer's disease. In: Biochemical Society Transactions.; 2011. doi:10.1042/BST0390898.
22. Kleinberger G, Yamanishi Y, Su.rez-Calvet M, et al. TREM2 mutations implicated in neurodegeneration impair cell surface transport and phagocytosis. Sci Transl Med. Published online 2014. doi:10.1126/scitranslmed.3009093.
23. Ho G J, Liang W, Waragai M, Sekiyama K, Masliah E, Hashimoto M. Bridging molecular genetics and biomarkers in Lewy body and related disorders. Int J Alzheimers Dis. Published online 2011. doi:10.4061/2011/842475
24. Henchcliffe C, Dodel R, Beal M F. Biomarkers of Parkinson's disease and S E, et al. Biomarker discovery for Alzheimer's disease, frontotemporal lobar degeneration, and Parkinson's disease. Acta Neuropathol. Published online 2010. doi:10.1007/s00401-010-0723-9.
25. Hu W T, Chen-Plotkin A, Arnold S E, et al. Biomarker discovery for Alzheimer's disease, frontotemporal lobar degeneration, and Parkinson's disease. Acta Neuropathol. Published online 2010. doi:10.1007/s00401-010-0723-9.
26. Heneka M T, Kummer M P, Latz E. Innate immune activation in neurodegenerative disease. Nat Rev Immunol. Published online 2014. doi:10.1038/nri3705.
27. Hakimi M, Selvanantham T, Swinton E, et al. Parkinson's disease-linked LRRK2 is expressed in circulating and tissue immune cells and upregulated following recognition of microbial structures. J Neural Transm. Published online 2011. doi:10.1007/s00702-011-0653-2.
28. Santiago J A, Scherzer C R, Potashkin J A. Network Analysis Identifies SOD2 mRNA as a Potential Biomarker for Parkinson's Disease. PLoS One. Published online 2014. doi:10.1371/journal.pone.0109042.
29. Li Y J, Oliveira S A, Xu P, et al. Glutathione S-transferase omega-1 modifies age-onset of Alzheimer disease and Parkinson disease. Hum Mol Genet. Published online 2003. doi:10.1093/hmg/ddg357.
30. Manocha G D, Floden A M, Puig K L, Nagamoto-Combs K, Scherzer C R, Combs C K. Defining the contribution of neuroinflammation to Parkinson's disease in humanized immune system mice. Mol Neurodegener. Published online 2017. doi:10.1186/s13024-017-0158-z.
31. Peter I, Dubinsky M, Bressman S, et al. Anti-tumor necrosis factor therapy and incidence of Parkinson disease among patients with inflammatory bowel disease. JAMA Neurol. Published online 2018. doi:10.1001/jamaneurol.2018.0605.
32. Olsen A L, Riise T, Scherzer C R. Discovering new benefits from old drugs with big data-promise for Parkinson disease. JAMA Neurol. Published online 2018. doi:10.1001/jamaneurol.2018.0345.
33. O'neill E, Harkin A. Targeting the noradrenergic system for anti-inflammatory and neuroprotective effects: Implications for Parkinson's disease. Neural Regen Res. Published online 2018. doi:10.4103/1673-5374.235219.
34. Wakade C, Giri B, Malik A, et al. Niacin modulates macrophage polarization in Parkinson's disease. J Neuroimmunol. Published online 2018. doi:10.1016/j.jneuroim.2018.05.002.
35. Mandal S, Das Mandal S, Chuttani K, Sawant K K, Subudhi B B. Preclinical study of ibuprofen loaded transnasal mucoadhesive microemulsion for neuroprotective effect in MPTP mice model. Iran J Pharm Res. Published online 2018. doi:10.22037/ijpr.2018.2169.
36. Joshi N, Singh S. Updates on immunity and inflammation in Parkinson disease pathology. J Neurosci Res. Published online 2018. doi:10.1002/jnr.24185.
37. Borzabadi S, Oryan S, Eidi A, et al. The effects of probiotic supplementation on gene expression related to inflammation, insulin and lipid in patients with Parkinson's disease: A randomized, double-blind, placebo-controlled trial. Arch Iran Med. Published online 2018.
38. Taghizadeh M, Jamilian M, Mazloomi M, Sanami M, Asemi Z. A randomized controlled clinical trial investigating the effect of omega-3 fatty acids and Vitamin E co-supplementation on markers of insulin metabolism and lipid profiles in gestational diabetes. J Clin Lipidol. Published online 2016. doi:10.1016/j.jacl.2015.12.017.
39. Shoulson I. DATATOP: a decade of neuroprotective inquiry. Parkinson Study Group. Deprenyl And Tocopherol Antioxidative Therapy Of Parkinsonism. Ann Neurol. Published online 1998.
40. Datatop: A multicenter controlled clinical trial in early Parkinson's disease: Parkinson study group. Arch Neurol. Published online 1989. doi:10.1001/archneur.1989.00520460028009.
41. Penney J B, Oakes D, Shoulson I, et al. Impact of deprenyl and tocopherol treatment on Parkinson's disease in DATATOP patients requiring levodopa. Ann Neurol. Published online 1996. doi:10.1002/ana.410390107.
42. Maki-Ikola O, Heinonen E, Penney J B, Shoulson I, Kieburtz K, Oakes D. Study design problems of DATATOP study analysis [2]. Ann Neurol. Published online 1996. doi:10.1002/ana.410400624.
43. LeWitt P, Oakes D, Cui L. The need for levodopa as an end point of Parkinson's disease progression in a clinical trial of selegiline and α-tocopherol. Mov Disord. Published online 1997. doi:10.1002/mds.870120208.
44. Liu C, Cholerton B, Shi M, et al. CSF tau and tau/Aβ42 predict cognitive decline in Parkinson's disease. Park Relat Disord. Published online 2015. doi:10.1016/j.parkreldis.2014.12.027.
45. Huang X, Auinger P, Eberly S, et al. Serum cholesterol and the progression of Parkinson's disease: Results from DATATOP. PLoS One. Published online 2011. doi:10.1371/journal.pone.0022854.
46. Shulman L M, Gruber-Baldini A L, Anderson K E, Fishman P S, Reich S G, Weiner W J. The clinically important difference on the unified Parkinson's disease rating scale. Arch Neurol. Published online 2010. doi: 10.1001/archneurol.2009.295.
47. O'Bryant S E, Xiao G, Zhang F, et al. Validation of a serum screen for Alzheimer's disease across assay platforms, species, and tissues. J Alzheimer's Dis. Published online 2014. doi:10.3233/JAD-141041.
48. O'Bryant S E, Edwards M, Johnson L, et al. A blood screening test for Alzheimer's disease. Alzheimer's Dement Diagnosis, Assess Dis Monit. Published online 2016. doi:10.1016/j.dadm.2016.06.004.
49. O'Bryant S E, Edwards M, Zhang F, et al. Potential two-step proteomic signature for Parkinson's disease: Pilot analysis in the Harvard Biomarkers Study. Alzheimer's Dement Diagnosis, Assess Dis Monit. Published online 2019. doi:10.1016/j.dadm.2019.03.001.
50. O'Bryant S E, Ferman T J, Zhang F, et al. A proteomic signature for dementia with Lewy bodies. Alzheimer's Dement Diagnosis, Assess Dis Monit. Published online 2019. doi:10.1016/j.dadm.2019.01.006.
51. Aisen P S, Schafer K A, Grundman M, et al. Effects of Rofecoxib or Naproxen vs Placebo on Alzheimer Disease Progression: A Randomized Controlled Trial. J Am Med Assoc. Published online 2003. doi:10.1001/jama.289.21.2819.
52. Applications Covered by Section 505(b)(2)|FDA. Accessed Dec. 20, 2020. www.fda.gov/regulatory-information/search-fda-guidancedocuments/applications-covered-section-505b2.

1. A method for selecting a subject for a targeted treatment for Parkinson's Disease (PD), the method comprising:
obtaining or having obtained a blood, plasma or serum sample from the subject;
measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP);
using a machine learning algorithm comparing the level of expression of the one or more first biomarkers from the subject's sample with the expression level of a corresponding one or more first biomarkers from a statistical sample representative of biomarkers in a normal subject; and
if the subject is found to have a statistically different expression level of the one or more biomarkers when compared to the expression level of the corresponding one or more biomarkers from the statistical sample, treating the subject with a monoamine oxidase inhibitor, an antioxidant, or both.

2. The method of claim 1, wherein the subject is categorized as a responder or other, wherein those subjects categorized as other include a non-responder or an adverse responder, wherein only a responder is treated with the monoamine oxidase inhibitor, the antioxidant, or both.

3. The method of claim 1, wherein the monoamine oxidase inhibitor is selected from at least one of: hydrazine, isocarboxazid, hydracarbazine, phenelzine, tranylcypromine, bifemelane, moclobemide, pirlindole, toloxatone, rasagiline, selegiline, safinamide, linezolid, or methylene blue, and salts and combinations thereof.

4. The method of claim 1, further comprising if the subject has the statistically different expression level of the one or more first biomarkers the subject will need treatment with L-DOPA in 15 months or less, or
if the subject has the statistically different expression level of the one or more first biomarkers treating the subject with the monoamine oxidase inhibitor, the antioxidant, or both to delay a treatment with L-DOPA.

5. (canceled)

6. The method of claim 1, further comprising measuring one or more second biomarkers in the blood, plasma or serum sample, wherein a second statistically different expression level of one or more second biomarkers selected from the group consisting of: A040, A042, total-tau, Neurofilament Light (Nf-L) and α-synuclein is indicative that the subject will respond to a treatment with the monoamine oxidase inhibitor, the antioxidant, or both.

7. The method of claim 1, wherein the antioxidant is selected from at least one of: ascorbic acid, phenolic acids, sorbic acid, sodium bisulfite, sodium metabisulfite, acetyl cysteine, sodium thiosulfate, ethylene diamine tetraacetic acid, sodium nitrite, ascorbyl stearate, ascorbyl palmitate, sodium ascorbate, monosterol citrate, alpha-thioglycerol, erythorbic acid, cysteine hydrochloride, citric acid, vitamin A, beta-carotene, tocopherol or vitamin E, tocopherol acetate, dibutylhydroxytoluene, soybean lecithin, sodium thioglycolate, butylhydroxyanisole, propyl gallate, tertiary butylhydroquinone, butylated hydroxyanisole, uric acid, cysteine, glutathione, lipoic acid, anthocyanidins, co-enzyme Q10, selenium, melatonin, thiourea, and salts and combinations thereof.

8. The method of claim 1, wherein the method further comprises the step of delaying a treatment for Parkinson's disease with levodopa; or
avoiding, not commencing, or discontinuing a treatment for Parkinson's disease, wherein the treatment is providing levodopa.

9. (canceled)

10. The method of claim 1, wherein the one or more first biomarkers are selected in order, wherein the expression level of 2, 3, 4, 5, 6, 7, 8, 9 10, 11, 12, 13, 14, 15, 16, 17, or 18 of the first biomarker are measured, or both.

11. (canceled)

12. The method of claim 1, wherein at least one of:
the subject responds to an anti-oxidant treatment to delay a need for levodopa for 15 months if two or more first biomarkers are preferentially detected in the following order: eotaxin 3, Factor VII, alpha-2-microglobulin (A2M), IL-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP3);
the subject responds to an monoamine oxidase inhibitor treatment to delay a need for levodopa for 15 months if two or more first biomarkers are preferentially detected in the following order: thrombopoietin (TPO), soluble intracellular adhesion molecule (sICAM-1), C-reactive protein (CRP), IL-5, fatty acid binding protein (FABP3), IL-6, soluble vascular cell adhesion molecule 1 (sVCAM-1), alpha-2-microglobulin (A2M), serum amyloid A1 cluster (SAA), IL-7, IL-10, tumor necrosis factor alpha (TNFα), IL-18, tenacin C, eotaxin 3, Chemokine (C-C Motif) Ligand 17 (TARC), inflammatory cytokine I-309 (I-309), beta-2-microglobulin (B2M), and Factor VII; or
the subject responds to a monoamine oxidase inhibitor and an antioxidant treatment to delay a need for levodopa for 15 months if two or more first biomarkers are preferentially detected in the following order: thrombopoietin (TPO), IL-10, tenacin C, IL-6, serum amyloid A1 cluster (SAA), C-reactive protein (CRP), IL-5, beta-2-microglobulin (B2M), soluble intracellular adhesion molecule (sICAM-1), inflammatory cytokine I-309 (I-309), IL-18, fatty acid binding protein (FABP3), Factor VII, eotaxin 3, tumor necrosis factor alpha (TNFα), alpha-2-microglobulin (A2M), soluble vascular cell adhesion molecule 1 (sVCAM-1), IL-7, and Chemokine (C-C Motif) Ligand 17 (TARC).

13. (canceled)

14. (canceled)

15. A method for selecting a subject for a targeted treatment for Parkinson's disease (PD), the method comprising:
measuring, in a blood, plasma or serum sample obtained from the subject, an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP); and
if the subject has a modified expression of the one or more first biomarkers determining if the subject is categorized a responder, or other, wherein other is a non-responder or adverse responder, wherein if the patient is categorized as responder then treating the subject with a monoamine oxidase inhibitor, an antioxidant, or both, or
if the subject is categorized as other, then treating the subject with a PD therapy.

16. The method of claim 15, wherein the monoamine oxidase inhibitor is selected from at least one of: hydrazine, isocarboxazid, hydracarbazine, phenelzine, tranylcypromine, bifemelane, moclobemide, pirlindole, toloxatone, rasagiline, selegiline, safinamide, linezolid, or methylene blue, and salts and combinations thereof.

17. The method of claim 15, wherein the antioxidant is selected from at least one of: ascorbic acid, phenolic acids, sorbic acid, sodium bisulfite, sodium metabisulfite, acetyl cysteine, sodium thiosulfate, ethylene diamine tetraacetic acid, sodium nitrite, ascorbyl stearate, ascorbyl palmitate, sodium ascorbate, monosterol citrate, alpha-thioglycerol, erythorbic acid, cysteine hydrochloride, citric acid, vitamin A, beta-carotene, tocopherol or vitamin E, tocopherol acetate, dibutylhydroxytoluene, soybean lecithin, sodium thioglycolate, butylhydroxyanisole, propyl gallate, tertiary butylhydroquinone, butylated hydroxyanisole, uric acid, cysteine, glutathione, lipoic acid, anthocyanidins, co-enzyme Q10, selenium, melatonin, thiourea, and salts and combinations thereof.

18. The method of claim 15, further comprising if the subject has a modified expression of the one or more first biomarkers and the subject is a non-responder or adverse responder the subject will need treatment with L-DOPA in 15 months or less; or if the subject has a modified expression of the one or more first biomarkers and the subject is a responder then treatment with the monoamine oxidase inhibitor, the antioxidant, or both is provided to delay a treatment with L-DOPA.

19. (canceled)

20. The method of claim 15, further comprising measuring one or more second biomarkers in the blood, plasma or serum sample, wherein a modified expression level of the one or more second biomarkers selected from the group consisting of: A040, A042, total-tau, Neurofilament Light (Nf-L) and α-synuclein is indicative that the subject will respond to a treatment with the monoamine oxidase inhibitor, the antioxidant, or both; or
measuring in the blood or serum sample an expression level of one or more second biomarkers selected from the group consisting of: Aβ40, Aβ42, total-tau, Neurofilament Light (Nf-L) and α-synuclein.

21. (canceled)

22. The method of claim 15, wherein the method further comprises the step of avoiding, not commencing, or discontinuing the treatment for Parkinson's disease with the monoamine oxidase inhibitor, the antioxidant, or both, and then providing levodopa; or
avoiding, not commencing, or discontinuing a treatment for Parkinson's disease, wherein the treatment is providing levodopa.

23. (canceled)

24. The method of claim 15, wherein the one or more first biomarkers are selected in order, wherein the expression level of 2, 3, 4, 5, 6, 7, 8, 9 10, 11, 12, 13, 14, 15, 16, 17, or 18 of the first biomarker are measured, or both.

25. (canceled)

26. The method of claim 15, wherein at least one of:
the subject responds to an anti-oxidant treatment to delay a need for levodopa for 15 months if two or more first biomarkers are preferentially detected in the following order: eotaxin 3, Factor VII, alpha-2-microglobulin (A2M), IL-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP3);
the subject responds to an monoamine oxidase inhibitor treatment to delay a need for levodopa for 15 months if two or more first biomarkers are preferentially detected in the following order: thrombopoietin (TPO), soluble intracellular adhesion molecule (sICAM-1), C-reactive protein (CRP), IL-5, fatty acid binding protein (FABP3), IL-6, soluble vascular cell adhesion molecule 1 (sVCAM-1), alpha-2-microglobulin (A2M), serum amyloid A1 cluster (SAA), IL-7, IL-10, tumor necrosis factor alpha (TNFα), IL-18, tenacin C, eotaxin 3, Chemokine (C-C Motif) Ligand 17 (TARC), inflammatory cytokine I-309 (I-309), beta-2-microglobulin (B2M), and Factor VII; or
the subject responds to a monoamine oxidase inhibitor and an antioxidant treatment to delay a need for levodopa for 15 months if two or more first biomarkers are preferentially detected in the following order: thrombopoietin (TPO), IL-10, tenacin C, IL-6, serum amyloid A1 cluster (SAA), C-reactive protein (CRP), IL-5, beta-2-microglobulin (B2M), soluble intracellular adhesion molecule (sICAM-1), inflammatory cytokine I-309 (I-309), IL-18, fatty acid binding protein (FABP3), Factor VII, eotaxin 3, tumor necrosis factor alpha (TNFα), alpha-2-microglobulin (A2M), soluble vascular cell adhesion molecule 1 (sVCAM-1), IL-7, and Chemokine (C-C Motif) Ligand 17 (TARC).

27. (canceled)

28. (canceled)

29. A method of identifying a Parkinson's Disease patient that will respond to a treatment that delays the need for L-DOPA comprising:
obtaining a blood, plasma or serum sample from the patient;
measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP);
using a machine learning algorithm comparing the level of expression from the blood, plasma or serum sample with a statistical sample representative of a normal subject; and
if the patient has a statistically different expression level of the one or more biomarkers when compared to the expression level of a corresponding one or more biomarkers from the statistical sample, treating the patient with a monoamine oxidase inhibitor, an antioxidant, or both to delay the treatment with L-DOPA.

30. A method of predicting and delaying or treating a loss of motor function, a loss of cognition, or both in a PD patient response to therapy comprising:
obtaining or having obtained a blood, plasma or serum sample from the patient;
measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP);
using a machine learning algorithm comparing the level of expression from the sample with a statistical sample representative of a normal subject;
determining if the patient has a statistically different expression level of the one or more biomarkers when compared to the expression level of a corresponding one or more biomarkers from the statistical sample; and
treating the patient having the statistically different expression level of the one or more biomarkers with a monoamine oxidase inhibitor, an antioxidant, or both, to delay or treat the loss of motor function, the loss of cognition, or both.

31. A method of identifying a subject with Parkinson's disease (PD) who will not respond to a monoamine oxidase inhibitor therapy, an antioxidant therapy, or a combination thereof, the method comprising:
obtaining or having obtained a blood, plasma or serum sample from the subject;
measuring in the blood, plasma or serum sample an expression level of one or more first biomarkers selected from the group consisting of: eotaxin 3, Factor VII, alpha-2-microglobulin (A2M), interleukin (IL)-10, inflammatory cytokine I-309 (I-309), soluble intracellular adhesion molecule (sICAM-1), C-reactive protein (CRP), tumor necrosis factor alpha (TNFα), thrombopoietin (TPO), IL-5, tenacin C, IL-18, IL-6, Chemokine (C-C Motif) Ligand 17 (TARC), soluble vascular cell adhesion molecule 1 (sVCAM-1), serum amyloid A1 cluster (SAA), beta-2-microglobulin (B2M), IL-7, and fatty acid binding protein (FABP);
using a machine learning algorithm comparing the level of expression from the sample with a statistical sample representative of a normal subject;
determining if the subject has a statistically different expression level of the one or more biomarkers when compared to the expression level of a corresponding one or more biomarkers from the statistical sample, wherein the statistically different expression level is indicative that the subject is categorized as a responder to a treatment with the monoamine oxidase inhibitor therapy, the antioxidant therapy, or a combination thereof, or the patient is categorized as a non-responder or an adverse responder; and
if the subject is categorized as non-responder or adverse responder, treating the subject with a traditional PD therapy selected from levodopa, carbidopa-levodopa, dopamine agonists, catechol O-methyltransferase inhibitors, anticholinergics, or amantadine.
